SEQUENCE LISTING (3 sequences)

SEQ ID NO: 1 - DNA, 18 bp, Artificial Sequence
  Forward primer
tgtatgaagaaggcttcg

SEQ ID NO: 2 - DNA, 19 bp, Artificial Sequence
  Reverse primer
aaaggtattaactttactc

SEQ ID NO: 3 - DNA, 20 bp, Artificial Sequence
  Probe
gtactttcagcggggaggaa

(19) United States
(12) Patent Application Publication
(10) Pub. No.: US 2009/0142798 A1
(43) Pub. Date: Jun. 4, 2009

(54) METHOD OF SELECTIVELY LYSING NON-VIABLE CELLS IN CELL POPULATION IN SAMPLE

(75) Inventors: Young-sun LEE, Yongin-si (KR); Myo-yong Lee, Suwon-si (KR); Soo-hyung Choi, Seoul (KR); Kak Namkoong, Seoul (KR); Chin-sung Park, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: Samsung Electronics Co., Ltd., Suwon-si (KR)

(21) Appl. No.: 12/108,586

(22) Filed: Apr. 24, 2008

(30) Foreign Application Priority Data
Dec. 4, 2007 (KR) 10-2007-0124903

(51) Int. Cl. G01N 33/48; C12N 1/06

(52) U.S. Cl. 435/40.5; 435/259

(57) ABSTRACT

The present invention provides a method of selectively lysing non-viable cells from a cell population within a sample, the method including selectively lysing non-viable cells by mixing a cell-containing sample with a lysis buffer including a non-ionic surfactant and a divalent cation salt.

### CROSS-REFERENCE TO RELATED PATENT APPLICATION

This application claims the benefit of Korean Patent Application No. 10-2007-0124903, filed on Dec. 4, 2007, in the Korean Intellectual Property Office, the disclosure of which is incorporated herein in its entirety by reference.

### BACKGROUND OF THE INVENTION

1. Field of the Invention

The present invention relates to a method of selectively lysing non-viable cells from a cell population within a sample and a method of selectively analyzing non-viable or viable cells from a cell population within a sample.

2. Description of the Related Art

There are cases where only viable cells must be analyzed within a sample. Examples of such cases include monitoring food and water safety, verifying sterility of pharmaceuticals, clinical diagnosis, and analyses of bioterroristic materials. When non-viable cells exist in such analyses above, the analyses may present false positive results.

Moreover, even when species in Campylobacter genus such as Campylobacter jejuni are alive, cell culture is not possible or requires a special condition for culture, thereby making it difficult to analyze the cells by culturing.

Conventional methods of distinguishing non-viable and viable cells include using a dye specifically permeating non-viable or viable cells, such as propidium iodide (PI) or SYT09, and using flow cytometry. For example, U.S. Pat. No. 7,262,009 discloses a method of excluding dead cells from analysis of a cell population within a sample, the method including contacting a sample with a viability probe which modifies the nucleic acid of dead cells within the sample, wherein the viability probe reacts selectively to nucleic acids from the dead cells, such that nucleic acids from viable cells remain unmodified by the viability probe; lysing the cells and mixing the nucleic acids from both viable and dead cells; and detecting the nucleic acids of the viable cells within the sample, wherein the detection includes hybridizing at least one oligonucleotide probe to a target sequence within the nucleic acids.

Notwithstanding the above-described conventional method, a method of selectively analyzing viable cells within a sample including both viable and non-viable cells is still in demand.

### SUMMARY OF THE INVENTION

The present invention provides a method of selectively lysing non-viable cells from a sample containing viable cells and non-viable cells.

The present invention also provides a method of selectively analyzing non-viable cells or viable cells from a sample containing viable cells and non-viable cells.

According to an aspect of the present invention, there is provided a method of selectively lysing non-viable cells from a cell population within a sample, the method comprising, selectively lysing non-viable cells by mixing a cell-containing sample with a lysis buffer comprising a non-ionic surfactant and a divalent cation salt.

According to another aspect of the present invention, there is provided a method of selectively analyzing non-viable cells or viable cells from a cell population within a sample, the method comprising:

selectively lysing non-viable cells by mixing a cell-containing sample with a lysis buffer comprising a non-ionic surfactant and a divalent cation salt; and

separating the lysed non-viable cells and viable cells.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The above and other features and advantages of the present invention will become more apparent by describing in detail exemplary embodiments thereof with reference to the attached drawings in which:

FIGS. 1A and 1B are electrophotograms illustrating a degree of lysis of non-viable cells and viable cells with respect to lysis buffers;

FIG. 2 is a graph illustrating the effects of each component in lysis buffer 1 on non-viable cells and viable cells;

FIG. 3 is a set of images observed with a fluorescent microscope, representing a mixture solution of non-viable cells and viable cells, a non-viable cell solution, and a viable cell solution, before and after treating with a lysis buffer;

FIG. 4 is a graph illustrating the lysing effects of TritonX 100 in a lysis buffer on non-viable cells and viable cells;

FIG. 5 is a graph illustrating the lysing effects when TritonX 100 was replaced with Tween 20, on non-viable cells and viable cells; and

FIG. 6 is a graph illustrating the lysing effects of MgCl₂ in a lysis buffer on non-viable cells and viable cells.

### DETAILED DESCRIPTION OF THE INVENTION

Hereinafter, the present invention will be described more fully with reference to the accompanying drawings, in which preferred embodiments of the invention are shown.

As used herein, the term “viable cell” refers to a cell who can proliferate in a suitable medium and the term “non-viable cell” refers to a cell who can not proliferate in a suitable medium. An example of non-viable cells includes lysed cell by heating, enzymatic activity, sonication, freezing, thawing etc.

According to an aspect of the present invention, the present invention provides a method of selectively lysing non-viable cells from a cell population within a sample, including selectively lysing non-viable cells by mixing a cell-containing sample with a lysis buffer including a non-ionic surfactant and a divalent cation salt.

The non-ionic surfactant may be selected from the group consisting of polyethylene ethers (TritonX 100, TritonX 102), Tween 20, octylphenol-ethylene oxide (NP40), and Brij 58 (polyoxyethylene acyl ether), but is not limited thereto. Preferably, TritonX 100 may be used.

The concentration of the non-ionic surfactant may be 0.1 vol % to 3 vol % The divalent cation salt may be Ca²⁺ salt or Mg²⁺ salt. More particularly, the divalent cation salt may be one of CaCl₂, MgCl₂ and MgSO₄.

The concentration of the divalent cation salt may be 1 mM to 25 mM.

According to an embodiment of the present invention, the non-ionic surfactant may be TritonX 100 with a concentration of 0.1 vol % to 3 vol %. The divalent cation salt may be MgCl₂ with a concentration of 1 mM to 25 mM.

The mixing may be performed at room temperature, for example, at 25° C. to 27° C. for 5 to 10 minutes, but is not limited thereto. Preferably, the mixing may be performed for 10 minutes at room temperature. The mixing may be performed with or without stirring. Moreover, the mixing may be performed with a presence of a buffer which maintains the mixture at a pH of 7 to 8. Preferably, the buffer may be Tris-HCl buffer with a concentration of 10 mM to 60 mM.

In the method of selectively lysing non-viable cells from a cell population within a sample according to the present invention, the cells may be gram-negative bacteria, gram-positive bacteria, or animal cells. Preferably, the cells may be gram-negative bacteria cells, and more preferably, E. coli.

According to an aspect of the present invention, the present invention also provides a method of selectively analyzing non-viable cells or viable cells from a cell population within a sample, the method including: selectively lysing non-viable cells by mixing a cell-containing sample with a lysis buffer including a non-ionic surfactant and a divalent cation salt; and separating the lysed non-viable cells from viable cells or vice verse.

The method of selectively analyzing non-viable cells or viable cells from a cell population within a sample according to the present invention includes selectively lysing non-viable cells by mixing a cell-containing sample with a lysis buffer including a non-ionic surfactant and a divalent cation salt.

The non-ionic surfactant may be selected from the group consisting of polyethylene ethers (TritonX 100, TritonX 102), Tween 20, octylphenol-ethylene oxide (NP40), and Brij 58 (polyoxyethylene acyl ether), but is not limited thereto. Preferably, TritonX 100 may be used.

The concentration of the non-ionic surfactant may be 0.1 vol % to 3 vol %.

The divalent cation salt may be Ca²⁺ salt or Mg²⁺ salt. More particularly, the divalent cation salt may be one of CaCl₂, MgCl₂ and MgSO₄.

The concentration of the divalent cation salt may be 1 mM to 25 mM.

According to an embodiment of the present invention, the non-ionic surfactant may be TritonX 100 with a concentration of 0.1 vol % to 3 vol %. The divalent cation salt may be MgCl₂ with a concentration of 1 mM to 25 mM.

The mixing may be performed at room temperature, for example, at 25° C. to 27° C. for 5 to 10 minutes, but is not limited thereto. Preferably, the mixing may be performed for 10 minutes at room temperature. The mixing may be performed with or without stirring. Moreover, the mixing may be performed with a presence of a buffer which maintains the mixture at a pH of 7 to 8. Preferably, the buffer may be Tris-HCl buffer with a concentration of 10 mM to 60 mM.

The method of selectively analyzing non-viable cells or viable cells from a cell population within a sample according to the present invention further includes separating the lysed non-viable cells from the viable cells, or vice verse.

The separation may be performed using a physical separation method such as centrifugation or filtration, or may be performed using an enzyme, for example, an enzyme that lyses target materials to be analyzed such as DNase and RNase, thereby selectively lysing the materials released from the lysed non-viable cells. However, the separation method is not limited thereto, and any conventional method of choice may be used. Preferably, the cells may be separated using centrifugation, into a supernatant including a non-viable cell lysate and a pellet including viable cells.

In the method of selectively analyzing non-viable cells or viable cells from a cell population within a sample according to the present invention, the cells may be gram-negative bacteria, gram-positive bacteria, or animal cells. Preferably, the cells may be gram-negative bacteria cells, and more preferably, E. coli.

The lysed non-viable cells or viable cells separated as such may be used for biological analyses. For example, nucleic acid amplifying processes such as PCR and RT-PCR, or hybridization may be performed on the nucleic acids obtained from the non-viable cells or viable cells. Moreover, the viable cells themselves may be cultured and used for analysis.

Hereinafter, the present invention will be described in more detail with reference to the following the examples. However, these examples are for illustrative purposes only, and are not intended to limit the scope of the present invention.

### EXAMPLE 1

### Effects of Lysis Buffer

In the present example, effects of selective lysis of non-viable cells and viable cells with respect to the types of the lysis buffer were verified. Lysis buffer 1 contained 0.8M sucrose, 2.5 vol % of TritonX 100, 5 mM of MgCl₂ and 30 mM of Tris HCl (pH 7.4), and Lysis buffer 2 contained 10 mM of KHCO₃, 155 mM of NH₄Cl, and 0.1 mM of EDTA, filter-sterilized using a 0.2 μm filter (Vogelstein, B. and Gillespie, D., Proc. Natl. Acad. Sci. USA 76, 615 (1979)).

Specifically, E. coli BL21 was inoculated in 10 mL of LB medium and cultured overnight, and then 1 mL of the culture was cultured in a new 10 mL medium for 2 hours, and the E. coli in its exponential phase was replaced to PBS to a concentration of 10⁸ cells/ml, and this was used as viable cells. For non-viable cells, the same volume of viable cells was heat-treated at 72° C. for 15 minutes (Journal of Microbiological Methods 67 (2006) 310-320). 0.1-0.3 ml of the viable cells and the non-viable cells obtained as such were each added to 1 mL of Lysis buffers 1 and 2, and were incubated for 10 minutes to lyse the cells at room temperatrue. Then, the cultures were centrifuged at 13,000 rpm for 30 seconds, taking the supernatant. 20 [symbol] of the supernatant taken from the culture was electrophoresed on 0.6% agarose gel. The electrophoresis was performed in 1× TBE buffer at 100V for 20 minutes.

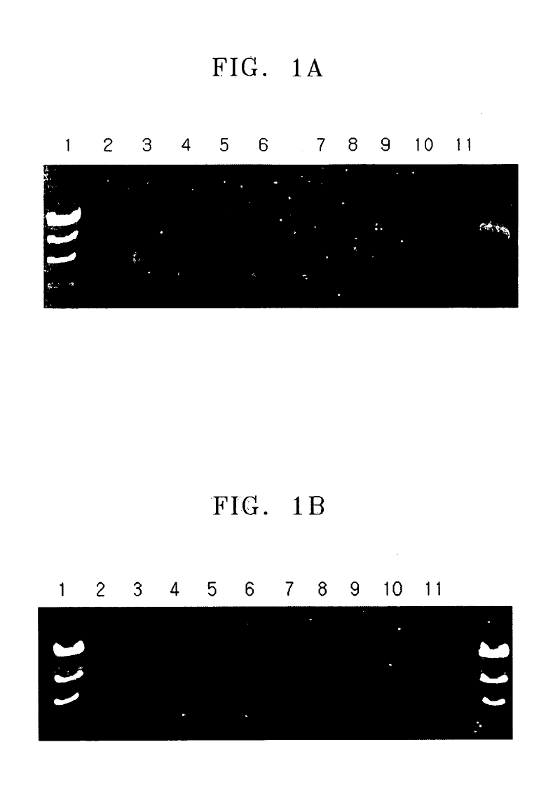

FIGS. 1A and 1B are electrophoretograms illustrating a degree of lysis of non-viable cells and viable cells with respect to lysis buffers.

Referring to FIG. 1A, when Lysis buffer 1 was used, DNA was observed in non-viable cells, but DNA was not observed in viable cells, showing that only the non-viable cells were lysed, while the viable cells were not lysed. In FIG. 1A, lane 1 represents a marker, (lambda HindIII marker, first size 25 kb), lanes 2 to 6 represent the results for non-viable cells, and lanes 7 to 11 represent the results for viable cells. The experiment was repeated 5 times.

Referring to FIG. 1B, when Lysis buffer 2 was used, DNA was not observed in either non-viable cells or viable cells, showing that the cells were not lysed. In FIG. 1B, lane 1 represents a marker, (lambda HindIII marker, first size 25 kb), lanes 2 to 6 represent the results for non-viable cells, and lanes 7 to 11 represent the results for viable cells. The experiment was repeated 5 times.

### EXAMPLE 2

### Effects of Each Components of the Lysis Buffer 1

In the present example, effects of selective lysis of non-viable cells with respect to the components of Lysis buffer 1 were verified. Lysis buffer 1 contained 0.8M sucrose, 2.5 vol % of TritonX 100, 5 mM of MgCl₂ and 30 mM of Tris HCl (pH 7.4).

Specifically, E. coli BL21 was inoculated in 10 mL of LB medium and cultured overnight, and then 1 mL of the culture was cultured in a new 10 mL medium for 2 hours, and the E. coli in its exponential phase was replaced to PBS to a concentration of 10⁸ cells/ml, and this was used as viable cells. For non-viable cells, the same volume of viable cells was heat-treated at 72° C. for 15 minutes (Journal of Microbiological Methods 67 (2006) 310-320). 0.1-0.3 ml of the viable cells and the non-viable cells obtained as such were each added to 1 mL of the Lysis buffer 1 and lysis buffers with each of sucrose, TritonX 100, MgCl₂, and Tris-HCl (pH 7.4) removed from Lysis buffer 1 (1× buffer), and were incubated for 10 minutes to lyse the cells. Then, the cultures were centrifuged at 13,000 rpm for 30 seconds, taking the supernatant. Taking 10 [symbol] of the supernatant taken from the culture as a template, and oligonucleotides of SEQ ID NOS. 1 and 2 (70 bp PCR product, forward primer: 5′-TGTATGAAGAAGGC TTCG-3′, reverse primer: 5′-AAAGGTATTAACTTTACTC-3′) as primers, PCR was performed. As a probe, an oligonucleotide of SEQ ID NO. 3 (5′-FAM-GTACTTTCAGCGGGGAGGAA-TMARA-3′) was used. 2× PCR master mix (1× PCR buffer, 5 mM MgCl₂, 250 nM dNTP, 0.1 U/ [symbol] Taq DNA polymerase (Solgent), 200 nM primers, 200 nM probe) was made and mixed with the supernatant DNA at 1:1 ratio to prepare the PCR solution. The PCR cycle included initial denaturation at 94° C. for 80 seconds, denaturation at 94° C. for 10 seconds, annealing at 50° C. for 30 seconds, and polymerization at 72° C. for 30 seconds, and was repeated 30 times. A real-time PCR was performed using Lightcycler 2 (Roche), and the degree of amplification was verified by measuring the Cp value (crossing point=Ct (threshold cycles). The cycle at which the amplification efficiency of PCR increases exponentially at 2n was measured, wherein n is a number of cycle. The effects of each components on cell lysis was determined by measuring the difference of the Cp values between the non-viable cells and the viable cells, that is, by measuring Δ Cp (unlysed cells □lysed cells).

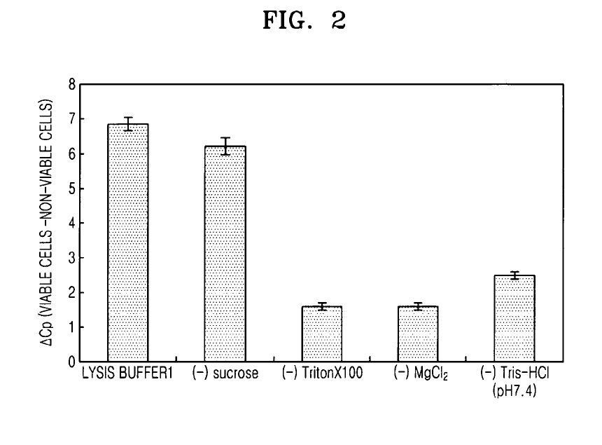

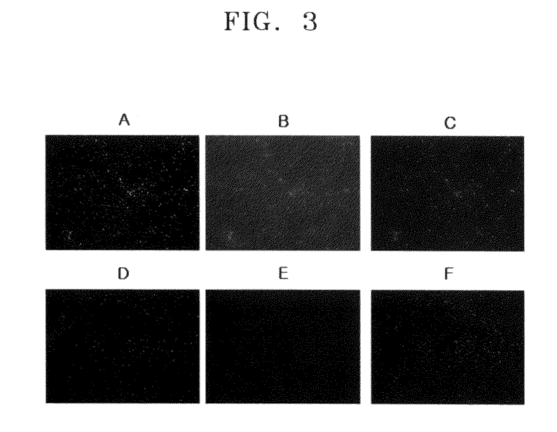

FIG. 2 is a graph illustrating the effects of each component in Lysis buffer 1 on non-viable cells and viable cells. Referring to FIG. 2, it can be seen that TritonX 100 and MgCl₂ are most effective in selectively lysing non-viable cells. Next, Tris-HCl (pH 7.4) affects the selective lysis of non-viable cells.

Tables 1 to 3 below respectively show the effects of TritonX 100, MgCl₂ and Tris-HCl (pH 7.4) on selectively lysing non-viable cells in terms of Cp values and Δ Cp values.

TABLE 1
Effects of TritonX 100 (lysis effect)

| | Viable cells | Non-viable cells |
|---|---|---|
| Cp | 30 | 28.385 |
| Cp | 1.615 | |

TABLE 2
Effects of MgCl₂ (cell protection effect)

| | Viable cells | Non-viable cells |
|---|---|---|
| Cp | 26.08 | 24.405 |
| Cp | 1.675 | |

TABLE 3
Effects of Tris-HCl (pH 7.4) (cell protection effect)

| | Viable cells | Non-viable cells |
|---|---|---|
| Cp | 27.36 | 24.865 |
| Cp | 2.495 | |

Referring to Table 1, since the Cp value of the viable cells and non-viable cell did not decrease when a cell lysis was conducted by using the solution without TritonX100, TritonX 100 is considered effective in lysing the cells. In addition, referring to Tables 2 and 3, since the Cp value of the viable cells decreased, MgCl₂ and Tris-HCl (pH 7.4) are considered to protect the cells. In particular, considering that Mg affects the Cp values for viable cells, it can be seen with regards to the stability of the cell membrane having a negative charge that even viable cells are lysed in the absence of MgCl₂, rendering a concentration control of Mg to an appropriate level important.

### EXAMPLE 3

### Verifying the Selective Lysis of Non-viable Cells by the Lysis Buffer

In the present example, it was verified through a fluorescent microscope that Lysis buffer 1 selectively lyses non-viable cells. Lysis buffer 1 contained 2.5 vol % of TritonX 100, 5 mM of MgCl₂ and 30 mM of Tris HCl (pH 7.4).

Specifically, E. Coli BL21 was inoculated in 10 mL of LB medium and cultured overnight, and then 1 mL of the culture was cultured in a new 10 mL medium for 2 hours, and the E. coli in its exponential phase was added to PBS to a concentration of 10⁸ cells/ml, which was used as viable cells. For non-viable cells, the same volume of viable cells was heat-treated at 72° C. for 15 minutes (Journal of Microbiological Methods 67 (2006) 310-320). 4.5 [symbol] of propidium iodide (PI) dye and SYTO9 dye were each added to 1 ml of a mixture of non-viable cells and viable cells obtained above, 1 ml of the non-viable cells, and 1 ml of the viable cells each (Invitrogen L7012, LIVE/NON-VIABLE™ BacLight™ Bacterial Viability Kits). These were incubated in the dark at room temperature for 15 minutes. Here, PI is a dye which does not permeate to viable cells, only dying non-viable cells with red color, and SYTO9 is a dye which permeates and dyes viable cells.

1 ml of the lysis buffer was added to 0.1 ml of each of the mixture solution, the non-viable cell solution, and the viable cell solution, and the cells were lysed by incubating for 10 minutes. Next, the incubi were observed with a fluorescent microscope.

FIG. 3 is a set of images observed with a fluorescent microscope, representing a mixture of non-viable cells and viable cells, a non-viable cell solution, and viable cell solution each treated with Lysis buffer 1. Referring to FIG. 3, it can be seen that all the red color of A and B has vanished in D and E. This shows that all the non-viable cells were lysed as a result of treating with Lysis buffer 1. In contrast, the green color of C is still present in F, showing that the viable cells were not lysed upon treatment with Lysis buffer 1. In FIG. 3, A, B, and C are fluorescent images representing the cell mixture solution, the non-viable cell solution and the viable cell solution, respectively, before the treatment with Lysis buffer 1, and D, E, and F, are fluorescent images representing the cell mixture solution, the non-viable cell solution and the viable cell solution, respectively, after the treatment with Lysis buffer 1.

### EXAMPLE 4

### Determination of Optimal Concentration of Each Component in Lysis Buffer 1

In the present example, the effect of different concentrations or replacements of TritonX 100 and MgCl₂ in Lysis buffer 1 in selectively lysing non-viable cells was verified. Lysis buffer 1 contained 0.8M sucrose, 2.5 vol % of TritonX 100, 12.5 mM of MgCl₂ and 30 mM of Tris HCl (pH 7.4).

Specifically, E. coli BL21 was inoculated in 10 mL of LB medium and cultured overnight, and then 1 mL of the culture was cultured in a new 10 mL medium for 2 hours, and the E. coli in its exponential phase was added to PBS to a concentration of 10⁸ cells/ml, which was used as viable cells. For non-viable cells, the same volume of viable cells was heat-treated at 72° C. for 15 minutes (Journal of Microbiological Methods 67 (2006) 310-320).

0.1 ml of the non-viable cells and the viable cells obtained as such were added to 1 ml each of a buffer obtained by varying the concentration of TritonX 100 within the range of 0 to 5 vol % from Lysis buffer 1, a buffer obtained by replacing the TritonX 100 in Lysis buffer 1 with Tween 20 in a range of 0 to 5 vol %, and a buffer obtained by varying the concentration of MgCl₂ within the range of 0 to 5 mM from Lysis buffer 1, and the cells were lysed by incubating for 10 minutes. Next, the incubi were centrifuged at 13,000 rpm for 30 seconds to obtain the supernatant. Taking 10 [symbol] of the obtained supernatant as a template, and oligonucleotides of SEQ ID NOS. 1 and 2 (70 bp PCR product, forward primer: 5′-TGTATGAAGAAGGC TTCG-3′, reverse primer: 5′-AAAGGTATTAACTTTACTC-3′) as primers, PCR was performed. As a probe, Taqman™ probe of an oligonucleotide of SEQ ID NO. 3 (5′-FAM-GTACTTTCAGCGGGGAGGAA-TMARA-3′) was used. 2× PCR master mix (1× PCR buffer, 5 mM MgCl₂, 250 nM dNTP, 0.1 U/ [symbol] Taq DNA polymerase (Solgent), 200 nM primers, 200 nM probe) was made and mixed with the supernatant DNA at 1:1 ratio to prepare the PCR solution. The PCR cycle included initial denaturation at 94° C. for 120 seconds, denaturation at 94° C. for 10 seconds, annealing at 50° C. for 30 seconds, and polymerization at 72° C. for 30 seconds, and was repeated 30 times. A real-time PCR was performed using Lightcycler 2 (Roche), and the degree of amplification was verified by measuring the Cp value (crossing point=Ct (threshold cycles). The cycle at which the amplification efficiency of PCR increases exponentially at 2n was measured wherein n is a number of cycles. The effects of each components on cell lysis was determined by measuring the difference of the Cp values between the non-viable cells and the viable cells, that is, by measuring Δ Cp (unlysed cells □lysed cells). For a positive control of non-viable cells, viable cells heat-treated at 95° C. for 15 minutes were used (completely lysed cells).

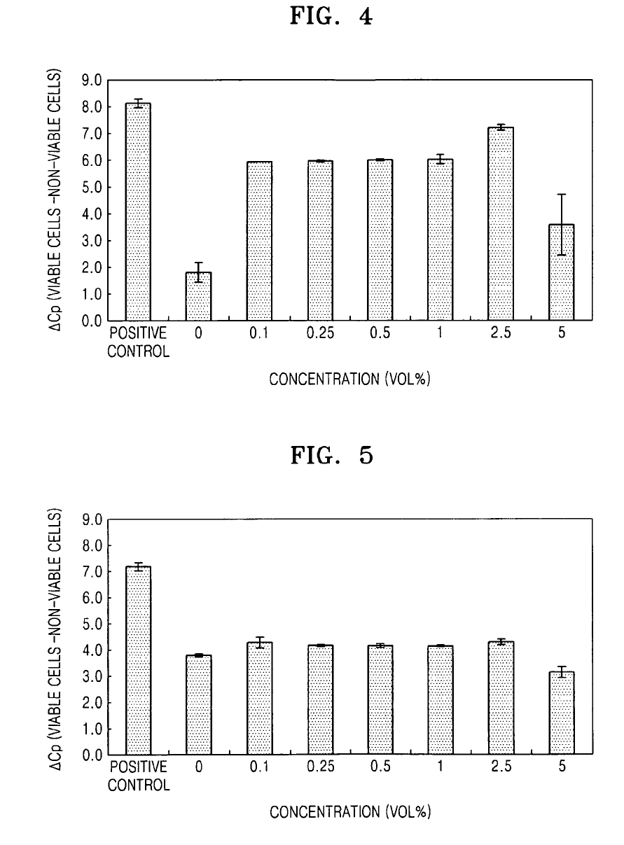

FIG. 4 is a graph illustrating the lysing effects of TritonX 100 in Lysis buffer 1 on non-viable cells and viable cells. Referring to FIG. 4, TritonX 100 was considered to be effective within the range of 0.1 vol % to 3 vol %.

FIG. 5 is a graph illustrating the lysing effects when TritonX 100 was replaced with Tween 20, on non-viable cells and viable cells. Referring to FIG. 5, Tween 20 was considered to be effective within the range of 0.1 vol % to 3 vol %. The untreated cells die out naturally during treatment, and therefore the Cp value difference is relatively small compared to other controls. Considering the controls for the same experiment, Tween 20 is effective.

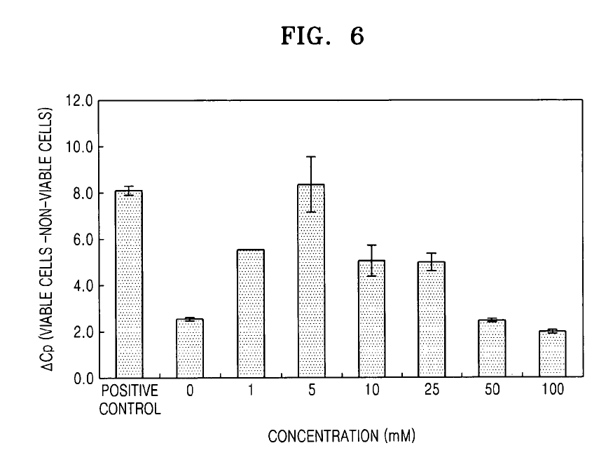

FIG. 6 is a graph illustrating the lysing effects of MgCl₂ in Lysis buffer 1 on non-viable cells and viable cells. Referring to FIG. 6, MgCl₂ was considered effective within the range of 1 mM to 25 mM.

Besides the experiment above, the same experiment using SDS was carried out, but because SDS lyses viable cells, it was not effective in selectively lysing non-viable cells (Data not shown).

From the results in Example 4, the final lysis buffer concentration was selected to be 2.5 vol % (v/v) TritonX 100/5 mM MgCl₂/30 mM Tris-HCl (pH 7.4).

According to the method of selectively lysing non-viable cells from a cell population within a sample of the present invention, only the non-viable cells may be selectively lysed within the sample.

According to the method of selectively analyzing non-viable cells or viable cells from a cell population within a sample of the present invention, only the non-viable cells or viable cells may be selectively analysed.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of selectively lysing non-viable cells from a cell population within a sample, the method comprising, selectively lysing non-viable cells by mixing a cell-containing sample with a lysis buffer comprising a non-ionic surfactant and a divalent cation salt.

2. The method of claim 1, wherein the non-ionic surfactant is selected from the group consisting of TritonX 100, Tween 20, NP40, and Brij 58.

3. The method of claim 1, wherein the concentration of the non-ionic surfactant is 0.1 vol % to 3 vol %

4. The method of claim 1, wherein the divalent cation salt is Ca²⁺ salt or Mg²⁺ salt.

5. The method of claim 4, wherein the divalent cation salt is CaCl₂, MgCl₂ or MgSO₄.

6. The method of claim 4, wherein the concentration of the divalent cation salt is 1 mM to 25 mM.

7. The method of claim 1, wherein the non-ionic surfactant is TritonX 100 with a concentration of 0.1 vol % to 3 vol %, and the divalent cation salt is MgCl₂ with a concentration of 1 mM to 25 mM.

8. A method of selectively analyzing non-viable cells or viable cells from a cell population within a sample, the method comprising:
selectively lysing non-viable cells by mixing a cell-containing sample with a lysis buffer comprising a non-ionic surfactant and a divalent cation salt; and
separating the lysed non-viable cells from viable cells or vice verse.

9. The method of claim 8, wherein the non-ionic surfactant is selected from the group consisting of TritonX 100, Tween 20, NP40, and Brij 58.

10. The method of claim 8, wherein the concentration of the non-ionic surfactant is 0.1 vol % to 3 vol %.

11. The method of claim 8, wherein the divalent cation salt is Ca²⁺ salt or Mg²⁺ salt.

12. The method of claim 11, wherein the divalent cation salt is CaCl₂, MgCl₂ or MgSO₄.

13. The method of claim 8, wherein the concentration of the divalent cation salt is 1 mM to 25 mM.

14. The method of claim 8, wherein the non-ionic surfactant is TritonX 100 with a concentration of 0.1 vol % to 3 vol %, and the divalent cation salt is MgCl₂ with a concentration of 1 mM to 25 mM.
